(19) United States
(12) Patent Application Publication
(10) Pub. No.: US 2019/0247307 A1
(43) Pub. Date: Aug. 15, 2019

(54) FORMULATION OF L-CYSTEINE HYDROCHLORIDE AMENABLE TO TERMINAL STERILIZATION

(71) Applicant: AL Pharma, Inc., Oklahoma City, OK (US)

(72) Inventor: John Hofstetter, Nobel, OK (US)

(21) Appl. No.: 16/393,697

(22) Filed: Apr. 24, 2019

Related U.S. Application Data
(63) Continuation-in-part of application No. 16/256,759, filed on Jan. 24, 2019.
(60) Provisional application No. 62/622,269, filed on Jan. 26, 2018.

(51) Int. Cl. A61K 9/08; A61K 9/00; A61K 31/198

(52) U.S. Cl. CPC A61K 9/08; A61L 2/0023; A61K 31/198; A61K 9/0019

(57) ABSTRACT

Stable formulation of L-cysteine hydrochloride amenable to terminal sterilization; a terminally sterilized stable formulation of L-cysteine hydrochloride; a process of manufacturing a stable formulation of L-cysteine hydrochloride amenable to terminal sterilization; and a container comprising the formulations of the invention.

### FIELD OF THE INVENTION

The present invention is related to a stable formulation of L-cysteine hydrochloride amenable to terminal sterilization; a terminally sterilized stable formulation of L-cysteine hydrochloride; a process of manufacturing a stable formulation of L-cysteine hydrochloride amenable to terminal sterilization; and a container comprising the formulations of the invention.

### BACKGROUND OF THE INVENTION

It is a well-accepted principle that sterile drugs should be manufactured using aseptic processing only when terminal sterilization is not feasible. The reason for this approach is that lack of sterility assurance is the primary reason for drug recalls. Nearly all drugs recalled due to lack of sterility assurance in the last twenty years were produced via aseptic processing (i.e. sterile filtration). By contrast, the use of terminal sterilization has historically resulted in substantially lower sterility failures. Terminal sterilization is the process of sterilizing a product in its final container. While there are no absolute Food and Drug Administration standards for sterilization processes, pharmaceutical solutions are most commonly sterilized using a steam sterilization with a heating regimen at 121.1° C. for a period long enough to ensure a safe sterility assurance level of 10′, meaning one out of one million products may contain a single organism. While this may be an effective method for thermally stable compounds, this practice is counterproductive for some heat-sensitive active pharmaceutical ingredients (“APIs”). In these cases, the resulting solution may be sterile, but it is often plagued with an unacceptable increase in degradation products brought on by the excessive use of heat in the sterilization process. Furthermore, compositions containing heat-sensitive APIs are often not terminally sterilized to avoid this degradation.

L-Cysteine Hydrochloride Injection, as a heat sensitive product, has historically not been amenable to terminal sterilization. To the best of the inventors' knowledge, there is currently no commercially available L-cysteine Hydrochloride Injection that is amenable to terminal sterilization.

Therefore, there is a need in the art for a formulation of L-cysteine hydrochloride injection amenable to terminal sterilization, as well as for a terminally sterilized formulation of L-cysteine hydrochloride injection.

### SUMMARY OF THE INVENTION

In one embodiment, the present invention provides a stable liquid formulation comprising L-cysteine hydrochloride, wherein the formulation is amenable to terminal sterilization and wherein the formulation contains no more than 10 parts per million (ppm) of dissolved oxygen, more preferably no more than 5 ppm of dissolved oxygen, and even more preferably no more than 1 ppm of dissolved oxygen.

In one embodiment, the present invention provides a stable sterile liquid formulation comprising L-cysteine hydrochloride, wherein the formulation was terminally sterilized by heat.

The terms “composition” and “formulation” are used interchangeably throughout the application.

In one embodiment, the formulation is terminally sterilized at a temperature of between about 100° C. and about 150° C.; more preferably between about 115° C. and about 125° C., and most preferably at about 121° C.

In a preferred embodiment, the formulation is sterilized by steam.

In a preferred embodiment, the provided liquid formulation is substantially free of oxygen.

In a preferred embodiment, the term “substantially free of oxygen” means that the provided liquid formulation contains no more than 10 parts per million (ppm) of dissolved oxygen, more preferably no more than 5 ppm of dissolved oxygen, and even more preferably no more than 1 ppm of dissolved oxygen.

In a preferred embodiment, the provided formulation is in a form of a sterile non-pyrogenic parenteral solution.

In a preferred embodiment, the provided parenteral solution is suitable as an additive to crystalline amino acid injections immediately prior to administration to a patient in need thereof.

In one embodiment, the provided formulation may comprise excipients, including but not limited to HCl, sodium hydroxide, and water. In one embodiment, nitrogen is used as a processing aid to deaerate the solution and to reduce the oxygen partial pressure and dissolved oxygen concentration in the drug solution vials (gas phase and liquid phase).

In a preferred embodiment, the terminally steam sterilized L-cysteine hydrochloride formulation when stored at a storage temperature of between 20° C. and 40° C. for three months, preferably for six months, more preferably for nine months, more preferably for twelve months and even more preferably for twenty four months, does not contain more than 2% of cystine impurities. In a preferred embodiment, the storage temperature is a room temperature (i.e., between about 20° C. and about 25° C.).

The present invention is further directed to a container comprising a sterile pharmaceutical formulation of the present invention.

In a preferred embodiment, the container is a 10-mL single dose vial which contains non-pyrogenic sterile solution of Cysteine Hydrochloride, wherein the solution was terminally sterilized by heat, preferably by steam sterilization.

The present invention is also directed to a process of manufacturing a pharmaceutical composition amenable to terminal sterilization, wherein the composition comprises L-cysteine hydrochloride, the process comprising the following steps:
(i) adding a pharmaceutically acceptable vehicle to a batch container comprising:
   (a) a top side comprising an opening; and
   (b) a pressurizing means,
(ii) purging the batch container with nitrogen gas;
(iii) mixing the drug vehicle;
(iv) adding L-cysteine hydrochloride monohydrate to the pharmaceutically acceptable vehicle to create the pharmaceutical composition;
(v) mixing the pharmaceutical composition until the L-cysteine hydrochloride monohydrate is dissolved in the pharmaceutically acceptable vehicle;
(vi) sealing the opening of the top side of the batch container;
(vii) pressurizing the batch container with nitrogen gas;
(viii) optionally, sterilizing stoppers, preferably in sterilization bags, preferably at about 121° C. and preferably for 30 minutes;
(ix) optionally, sterilizing filling equipment, preferably at about 121° C., preferably for 30 minutes, and preferably wrapping filling equipment prior to sterilization;
(x) optionally, washing vials with water for injection and depyrogenating vials in a depyrogenation oven;
(xi) optionally, filling a product container with the pharmaceutical composition from the batch container using filling equipment;
(xii) optionally, stoppering the product container; and
(xiii) optionally, capping the product container.

In one embodiment, the process further includes step (xiv) of terminally sterilizing the composition by heat.

The present invention is further directed to a sterile pharmaceutical composition prepared by a process of the present invention.

### DETAILED DESCRIPTION OF THE INVENTION

L-Cysteine Hydrochloride Injection, as a heat sensitive product has historically not been amenable to terminal sterilization. L-Cysteine Hydrochloride Injection, USP is intended for use only after dilution as an additive to Crystalline Amino Acid Injections to meet the intravenous amino acid nutritional requirements of infants receiving total parenteral nutrition. In premixed solutions of crystalline amino acids, cysteine is relatively unstable over time, eventually converting to insoluble cystine in the presence of oxygen. To avoid such precipitation, L-Cysteine Hydrochloride Injection USP is intended to be used as an additive with crystalline Amino Acid Injections immediately prior to administration to the patient. L-Cysteine Hydrochloride Injection, USP is intended for use only after dilution as an additive to Crystalline Amino Acid Injections to meet the intravenous amino acid nutritional requirements of infants receiving total parenteral nutrition.

In one embodiment, the present invention provides a stable liquid formulation comprising L-cysteine hydrochloride, wherein the formulation is amenable to terminal sterilization, and wherein the formulation contains no more than 10 parts per million (ppm) of dissolved oxygen, more preferably no more than 5 ppm of dissolved oxygen, and even more preferably no more than 1 ppm of dissolved oxygen.

In one embodiment, the present invention provides a stable sterile formulation comprising L-cysteine hydrochloride, wherein the formulation was terminally sterilized by heat.

In one embodiment, the formulation is terminally sterilized at a temperature of between about 100° C. and about 150° C.; more preferably between about 115° C. and about 125° C.; and most preferably at about 121° C.

In a preferred embodiment, the formulation is sterilized by steam.

In a preferred embodiment, the provided formulation is substantially free of oxygen.

In a preferred embodiment, the term “substantially free of oxygen” means that the provided terminally sterilized composition contains no more than 10 parts per million (ppm) of dissolved oxygen, more preferably no more than 5 ppm of dissolved oxygen, and even more preferably no more than 1 ppm of dissolved oxygen.

In a preferred embodiment, the provided formulation is in a form of a sterile non-pyrogenic parenteral solution.

In a preferred embodiment, the provided parenteral solution is suitable as an additive to crystalline amino acid injections immediately prior to administration to a patient in need thereof.

In one embodiment, the provided formulation may comprise excipients, including but not limited to HCl, sodium hydroxide, and water. In one embodiment, nitrogen is used as a processing aid to deaerate the solution and to reduce the oxygen concentration in the drug solution vials (in both gas phase and liquid phase).

In another preferred embodiment, the terminally sterilized L-cysteine hydrochloride formulation when stored at a storage temperature of between 20° C. and 40° C. for three months, preferably for six months, more preferably for nine months, more preferably for twelve months and even more preferably for twenty four months, does not contain more than 2% of cystine impurities. In a preferred embodiment, the storage temperature is a room temperature (i.e., between about 20° C. and about 25° C.).

The present invention is further directed to a container comprising a sterile pharmaceutical formulation of the present invention.

In a preferred embodiment, the container is a 10-mL single dose vial which contains non-pyrogenic sterile solution of Cysteine Hydrochloride, wherein the solution was terminally sterilized by heat, preferably by steam sterilization.

In a preferred embodiment, sterile pharmaceutical compositions of the present invention have a pH from about 1.0 to about 2.5.

In another preferred embodiment, sterile pharmaceutical compositions of the present invention comprise less than about 1.2% cystine.

The L-cysteine Hydrochloride Injection contains L-Cysteine HCl monohydrate as the API. The invention encompasses all forms of L-cysteine HCl monohydrate, including all enantiomers, stereoisomers, hydrates, solvates and salts thereof.

The present invention is also directed to a process of manufacturing a pharmaceutical composition amenable to terminal sterilization, wherein the composition comprises L-cysteine hydrochloride, the process comprising the following steps:
(i) adding a pharmaceutically acceptable vehicle to a batch container comprising:
   (a) a top side comprising an opening; and
   (b) a pressurizing means,
(ii) purging the batch container with nitrogen gas;
(iii) mixing the drug vehicle;
(iv) adding L-cysteine hydrochloride monohydrate to the pharmaceutically acceptable vehicle to create the pharmaceutical composition;
(v) mixing the pharmaceutical composition until the L-cysteine hydrochloride monohydrate is dissolved in the pharmaceutically acceptable vehicle;
(vi) sealing the opening of the top side of the batch container;
(vii) pressurizing the batch container with nitrogen gas;
(viii) optionally, sterilizing stoppers, preferably in sterilization bags, preferably at about 121° C. and preferably for 30 minutes;
(ix) optionally, sterilizing filling equipment, preferably at about 121° C., preferably for 30 minutes, and preferably wrapping filling equipment prior to sterilization;
(x) optionally, washing vials with water for injection and depyrogenating vials in a depyrogenation oven;
(xi) optionally, filling a product container with the pharmaceutical composition from the batch container using filling equipment;
(xii) optionally, stoppering the product container; and
(xiii) optionally, capping the product container.

In one embodiment, the process further includes step (xiv) of terminally sterilizing the composition by heat.

In a preferred embodiment, the concentration of L-cysteine hydrochloride monohydrate, is at about 50 milligrams per milliliter.

In another preferred embodiment, the pharmaceutically acceptable vehicle is water for injection.

Optionally, the process includes one or more of the following steps following step (v) and prior to step (vi):

determining a dissolved oxygen content of the pharmaceutically acceptable vehicle after step (iii) and before step (iv);

removing a portion of the pharmaceutical composition through a pharmaceutical composition removal means on the bottom side of the batch container;

adding the removed pharmaceutical composition to the opening of the top side of the batch container;

adding additional pharmaceutically acceptable vehicle to the pharmaceutical composition to obtain a desired concentration of L-cysteine hydrochloride monohydrate; and

removing an amount of pharmaceutical composition necessary for assay and pH testing.

The present invention is further directed to a sterile pharmaceutical composition prepared by a process of the present invention.

Unless explicitly stated otherwise, the term “terminally sterilized” refers to terminal sterilization by heat.

As used herein, the terms “around,” “about” or “approximately” shall generally mean within 20 percent, within 10 percent, within 5, 4, 3, 2 or 1 percent of a given value or range. Numerical quantities given are approximate, meaning that the term “around,” “about” or “approximately” can be inferred if not expressly stated.

The terms “long-term storage” or “long term stability” are understood to mean that the pharmaceutical composition can be stored for three months or more, for six months or more, and preferably for one year or more, most preferably a minimum stable shelf life of at least two years. Generally speaking, the terms “long term storage” and “long term stability” further include stable storage durations that are at least comparable to or better that the stable shelf typically required for currently available commercial formulations of L-cysteine Hydrochloride Inj ecti on, without losses in stability that would render the formulation unsuitable for its intended pharmaceutical application. Long-term storage is also understood to mean that the pharmaceutical composition may be stored as a liquid at 2-8° C. or at a room temperature.

The term “stable” with respect to long-term storage is understood to mean that cystine impurities contained in the pharmaceutical compositions are less than about 2%, or more preferably less than about 1%.

The term “room temperature” refers to a temperature of between about 20° C. and about 25° C.

As used herein, the term “pharmaceutically acceptable” refers to ingredients that are not biologically or otherwise undesirable for administration to a living subject.

Pressurizing means include, but are not limited to, a port, valve or other opening capable of selectively inputting and outputting air pressure.

Removing means include, but are not limited to, a port, valve or other opening capable of selectively inputting and outputting a liquid composition.

Stoppers suitable for use in the present invention include, but are not limited to, rubber stoppers, plastic stoppers and stoppers made from a combination of metal, rubber and/or plastic.

Filling equipment is generally known in the industry.

Depyrogenation is a technique generally known in the industry.

The following examples are intended to illustrate the process of the present invention and to teach one of ordinary skill in the art how to use the process of the invention. They are not intended to be limiting in any way.

### EXAMPLES

### Example 1—A Process for Manufacturing Sterile L-Cysteine Hydrochloride Monohydrate Using Terminal Sterilization (Autoclaving)

Lines and equipment were checked and cleared prior to mixing a tank batch. The batch container was then labeled as “quarantine.” The batch container was tared on a scale and water for injection (“WFI”) was added to the appropriate amount. The batch container was then purged with nitrogen gas to remove oxygen from the container, head space and liquid phase. The liquid phase was sparged with nitrogen to reduce the oxygen levels in liquid phase and gas phase to the lowest levels possible. A mixer was then inserted into the batch container and the WFI was mixed at an appropriate speed. Oxygen content of the WFI was then determined. L-cysteine hydrochloride monohydrate was added to the WFI at an appropriate concentration to create a pharmaceutical composition. 3 liters of the pharmaceutical composition was then removed from the bottom of the batch container and added to the top of the batch container. WFI was then added to an appropriate weight to create a 50 mg/mL L-cysteine hydrochloride monohydrate. Following the dilution the pharmaceutical composition to the appropriate concentration, 100 mL samples were taken for assay and pH testing, of which data is shown in Example 2 below. The batch container is then sealed and pressurized with nitrogen gas to a 5 psi.

The pharmaceutical composition manufactured by the process above was then filled into vials. Prior to filling, stoppers were sterilized in ready to sterilize bags in an autoclave at 121° for 30 minutes. The sterilized bags are then transferred to the fill room and then to the fill line. Additionally, prior to filling, equipment used for filling the vials was wrapped and sterilized in an autoclave at 121° for 30 minutes and transferred to the filling room. Finally, prior to filling, vials washed both interiorly and exteriorly using WFI in machine washing equipment and then depyrogenated using the dry heat method in a depyrogenation oven. Filling equipment and lines were then cleared and checked. Vials were then filled and stoppered within class 100 (iso 5) conditions using the appropriate equipment. Fill volume levels were checked at appropriate intervals. Vials were then transferred to a capping area via a conveyer belt. Capped vials were then transferred to a traying area via a conveyor belt.

Vials were then loaded onto SS trays. Qualified vial inspectors then inspect vials for defects. Rejected products were then placed into labeled reject bins and passing product is transported to quarantine or directly to applicable vial labeling area.

The vials containing the drug product were then autoclaved (i.e., steam sterilized) at 121° C. to achieve a 6-log reduction in microbial kill.

### Example 2—Stability of L-Cysteine Hydrochloride Monohydrate Produces by a Process of the Invention

### Method

A 10-mL or a 50-mL drug product containing 50 mg/mL L-cysteine hydrochloride monohydrate and water for injection (“WFI”), produced by the process of Example 1, was stored at various accelerated storage conditions for up to 36 months. Samples were taken at 1, 2, 3, 6, 9 months and assayed for bacterial endotoxins, pH, heavy metals including aluminum, drug product assay, fill volume, particulate matter, sterility and impurities.

TABLE 1
Inverted 10-mL L-cysteine hydrochloride monohydrate at 40° C. ± 2° C. 75% RH ± 5%

| | | Result | | | |
|---|---|---|---|---|---|
| Test | Acceptance Criteria | 1 Month | 2 Months | 3 Months | 6 Months |
| pH | 1.0 to 2.5 | 1.3 | 1.2 | 1.2 | 1.2 |
| Assay | 85% to 115% label claim | 109.3% | 107.4% | 109.8% | 110.6% |
| Individual Impurity: | Known: ≥0.05% Report Results | 0.55% | 0.59% | 0.59% | 0.69% |
| Cystine | Cystine Impurity ≤2.0% | | | | |
| Total Impurity | Sum All Reported ≥0.05% | 0.7% | 0.8% | 0.8% | 0.9% |

TABLE 2
Inverted 50-mL L-cysteine hydrochloride monohydrate at 40° C. ± 2° C. 75% RH ± 5%

| | | Result | | | |
|---|---|---|---|---|---|
| Test | Acceptance Criteria | 1 Month | 2 Months | 3 Months | 6 Months |
| pH | 1.0 to 2.5 | 1.3 | 1.2 | 1.3 | 1.2 |
| Assay | 85% to 115% label claim | 108.5% | 107.4% | 107.9% | 108.4% |
| Individual Impurity: | Known: ≥0.05% Report Results | 1.02% | 0.89% | 0.97% | 1.18% |
| Cystine | Cystine Impurity ≤2.0% | | | | |
| Total Impurity | Sum All Reported ≥0.05% | 1.2% | 1.1% | 1.2% | 1.4% |

TABLE 3
Upright 10-mL L-cysteine hydrochloride monohydrate at 25°
C. ± 2° C. 60% RH ± 5%

| | | Result | | |
|---|---|---|---|---|
| | | 3 | 6 | 9 |
| Test | Acceptance Criteria | Month | Month | Month |
| pH | 1.0 to 2.5 | 1.2 | 1.2 | 1.2 |
| Assay | 85% to 115% label claim | 110.2% | 110.0% | 109.6% |
| Individual | Known: ≥0.05% Report | 0.58% | 0.80% | 0.74% |
| Impurity: | Results Cystine | | | |
| Cystine | Impurity ≤2.0% | | | |
| Total | Sum All | 0.7% | 0.9% | 0.8% |
| Impurity | Reported ≥0.05% | | | |

TABLE 4
Invert 10-mL L-cysteine hydrochloride monohydrate at 25°
C. ± 2° C. 60% RH ± 5%

| | | Result | | |
|---|---|---|---|---|
| | | 3 | 6 | 9 |
| Test | Acceptance Criteria | Month | Month | Month |
| pH | 1.0 to 2.5 | 1.2 | 1.2 | 1.2 |
| Assay | 85% to 115% label claim | 110.9% | 110.1% | 110.2% |
| Individual | Known: ≥0.05% Report | 0.53% | 0.55% | 0.55% |
| Impurity: | Results Cystine | | | |
| Cystine | Impurity ≤2.0% | | | |
| Total | Sum All | 0.6% | 0.7% | 0.7% |
| Impurity | Reported ≥0.05% | | | |

TABLE 5
Inverted 10-mL L-cysteine hydrochloride monohydrate at 30°
C. ± 2° C. 60% RH ± 5%

| | | Result | | |
|---|---|---|---|---|
| | | 3 | 6 | 9 |
| Test | Acceptance Criteria | Month | Month | Month |
| pH | 1.0 to 2.5 | 1.2 | 1.2 | 1.2 |
| Assay | 85% to 115% label claim | 108.9% | 110.7% | 109.0% |
| Individual | Known: ≥0.05% Report | 0.67% | 0.63% | 0.67% |
| Impurity: | Results Cystine | | | |
| Cystine | Impurity ≤2.0% | | | |
| Total | Sum All | 0.8% | 0.8% | 0.8% |
| Impurity | Reported ≥0.05% | | | |

### Results

As shown in Tables 1-5, the 10-mL and 50-mL drug product containing 50 mg/mL L-cysteine hydrochloride monohydrate and WFI was stable at all storage condition and durations. Thus, drug product produced by the process of the present invention is free of the steam sterilization issues that accompany heat sensitive active pharmaceutical ingredients.

### Example 3—Stability of L-Cysteine Hydrochloride Monohydrate Produced by a Process of the Invention

### Method

A 10-mL or a 50-ml drug product containing 50 mg/mL L-cysteine hydrochloride monohydrate and water for injection (“WFI”), produced by the process of Example 1, was stored at various accelerated storage conditions for up to 36 months. Samples were taken at 0, 3, 6, 9, 12, 18, 24 and 36 months and assayed for bacterial endotoxins, pH, heavy metals including aluminum, drug product assay, fill volume, particulate matter, sterility and impurities.

TABLE 6
Upright 10-mL L-cysteine hydrochloride monohydrate at 25° C. ± 2° C. 60% RH ± 5%

| | | Result (%) | | | | | | | |
|---|---|---|---|---|---|---|---|---|---|
| | Acceptance | months | | | | | | | |
| Test | Criteria | 0 | 3 | 6 | 9 | 12 | 18 | 24 | 36 |
| Assay | 85% to 115% | 109.7 | 109.3 | 110.3 | 108.5 | 108.6 | 109.3 | 107.9 | 107.7 |
| | label claim | | | | | | | | |
| Individual | Known: ≥0.05% | 0.48 | 0.57 | 0.65 | 0.68 | 0.72 | 0.75 | 0.79 | 0.84 |
| Impurity: | Report Results | | | | | | | | |
| Cystine | Cystine Impurity | | | | | | | | |
| | ≤2.0% | | | | | | | | |
| Total | Sum All | 0.5 | 0.7 | 0.8 | 0.8 | 0.8 | 0.9 | 0.9 | 1.0 |
| Impurity | Reported | | | | | | | | |
| | ≥0.05% | | | | | | | | |

TABLE 7
Inverted 10-mL L-cysteine hydrochloride monohydrate at 25° C. ± 2° C. 60% RH ± 5%

| | | Result (%) | | | | | | | |
|---|---|---|---|---|---|---|---|---|---|
| | Acceptance | months | | | | | | | |
| Test | Criteria | 0 | 3 | 6 | 9 | 12 | 18 | 24 | 36 |
| Assay | 85% to 115% | 109.7 | 110.9 | 110.1 | 110.2 | 108.6 | 108.5 | 108.3 | 106.5 |
| | label claim | | | | | | | | |
| Individual | Known: ≥0.05% | 0.48 | 0.53 | 0.55 | 0.55 | 0.71 | 0.73 | 0.73 | 0.86 |
| Impurity: | Report Results | | | | | | | | |
| Cystine | Cystine Impurity | | | | | | | | |
| | ≤2.0% | | | | | | | | |
| Total | Sum All | 0.5 | 0.6 | 0.7 | 0.7 | 0.8 | 0.9 | 0.9 | 1.1 |
| Impurity | Reported | | | | | | | | |
| | ≥0.05% | | | | | | | | |

TABLE 8
Upright 10-mL L-cysteine hydrochloride monohydrate at 25° C. ± 2° C. 60% RH ± 5%

| | | Result (%) | | | | | | | |
|---|---|---|---|---|---|---|---|---|---|
| | Acceptance | months | | | | | | | |
| Test | Criteria | 0 | 3 | 6 | 9 | 12 | 18 | 24 | 36 |
| Assay | 85% to 115% | 109.5 | 109.2 | 110.3 | 107.7 | 110.5 | 108.4 | 108.5 | 107.0 |
| | label claim | | | | | | | | |
| Individual | Known: ≥0.05% | 0.54 | 0.65 | 0.73 | 0.60 | 0.88% | 0.75 | 0.82 | 0.94 |
| Impurity: | Report Results | | | | | | | | |
| Cystine | Cystine Impurity | | | | | | | | |
| | ≤2.0% | | | | | | | | |
| Total | Sum All | 0.6 | 0.8 | 0.9 | 0.7 | 1.0 | 0.9 | 1.0 | 1.2 |
| Impurity | Reported | | | | | | | | |
| | ≥0.05% | | | | | | | | |

TABLE 9
Inverted 10-mL L-cysteine hydrochloride monohydrate at 25° C. ± 2° C. 60% RH ± 5%

| | | Result (%) | | | | | | | |
|---|---|---|---|---|---|---|---|---|---|
| | Acceptance | months | | | | | | | |
| Test | Criteria | 0 | 3 | 6 | 9 | 12 | 18 | 24 | 36 |
| Assay | 85% to 115% | 109.5 | 109.9 | 110.8 | 109.1 | 107.7 | 109.4 | 108.9 | 107.1 |
| | label claim | | | | | | | | |
| Individual | Known: ≥0.05% | 0.54 | 0.65 | 0.80 | 0.61 | 0.74% | 0.66 | 0.77 | 0.86 |
| Impurity: | Report Results | | | | | | | | |
| Cystine | Cystine Impurity | | | | | | | | |
| | ≤2.0% | | | | | | | | |
| Total | Sum All | 0.6 | 0.8 | 1.0 | 0.7 | 0.9 | 0.8 | 0.9 | 1.1 |
| Impurity | Reported | | | | | | | | |
| | ≥0.05% | | | | | | | | |

TABLE 10
Inverted 10-mL L-cysteine hydrochloride monohydrate at
30° C. ± 2° C. 65% RH ± 5%

| | | Result (%) | | | | |
|---|---|---|---|---|---|---|
| | Acceptance | months | | | | |
| Test | Criteria | 0 | 3 | 6 | 9 | 12 |
| Assay | 85% to | 109.5 | 109.6 | 111.6 | 108.6 | 108.0 |
| | 115% label | | | | | |
| | claim | | | | | |
| Individual | Known: | 0.54 | 0.64 | 0.68 | 0.62 | 0.92 |
| Impurity: | ≥0.05% | | | | | |
| Cystine | Report | | | | | |
| | Results | | | | | |
| | Cystine | | | | | |
| | Impurity | | | | | |
| | ≤2.0% | | | | | |
| Total | Sum All | 0.6 | 0.8 | 0.8 | 0.8 | 1.1 |
| Impurity | Reported | | | | | |
| | ≥0.05% | | | | | |

TABLE 11
Inverted 10-mL L-cysteine hydrochloride monohydrate at
40° C. ± 2° C. 75% RH ± 5%

| | | Result (%) | | | | |
|---|---|---|---|---|---|---|
| | Acceptance | months | | | | |
| Test | Criteria | 0 | 1 | 2 | 3 | 6 |
| Assay | 85% to | 109.5 | 109.3 | 107.2 | 109.6 | 111.6 |
| | 115% label | | | | | |
| | claim | | | | | |
| Individual | Known: | 0.54 | 0.57 | 0.75 | 0.70 | 0.88 |
| Impurity: | ≥0.05% | | | | | |
| Cystine | Report | | | | | |
| | Results | | | | | |
| | Cystine | | | | | |
| | Impurity | | | | | |
| | ≤2.0% | | | | | |
| Total | Sum All | 0.6 | 0.7 | 0.9 | 0.9 | 1.1 |
| Impurity | Reported | | | | | |
| | ≥0.05% | | | | | |

TABLE 12
Upright 10-mL L-cysteine hydrochloride monohydrate at 25° C. ± 2° C. 60% RH ± 5%

| | | Result (%) | | | | | | | |
|---|---|---|---|---|---|---|---|---|---|
| | Acceptance | months | | | | | | | |
| Test | Criteria | 0 | 3 | 6 | 9 | 12 | 18 | 24 | 36 |
| Assay | 85% to 115% | 109.4 | 110.2 | 110.0 | 109.6 | 108.0 | 109.7 | 109.3 | 108.1 |
| | label claim | | | | | | | | |
| Individual | Known: ≥0.05% | 0.45 | 0.58 | 0.80 | 0.74 | 0.67 | 0.69 | 0.75 | 0.83 |
| Impurity: | Report Results | | | | | | | | |
| Cystine | Cystine Impurity | | | | | | | | |
| | ≤2.0% | | | | | | | | |
| Total | Sum All | 0.5 | 0.7 | 0.9 | 0.8 | 0.8 | 0.9 | 0.9 | 1.0 |
| Impurity | Reported | | | | | | | | |
| | ≥0.05% | | | | | | | | |

TABLE 13
Inverted 10-mL L-cysteine hydrochloride monohydrate at 25° C. ± 2° C. 60% RH ± 5%

| | | Result (%) | | | | | | | |
|---|---|---|---|---|---|---|---|---|---|
| | Acceptance | months | | | | | | | |
| Test | Criteria | 0 | 3 | 6 | 9 | 12 | 18 | 24 | 36 |
| Assay | 85% to 115% | 109.4 | 109.6 | 112.5 | 110.0 | 108.3 | 109.0 | 110.2 | 107.6 |
| | label claim | | | | | | | | |
| Individual | Known: ≥0.05% | 0.45 | 0.53 | 0.69 | 0.66 | 0.67 | 0.69 | 0.89 | 0.77 |
| Impurity: | Report Results | | | | | | | | |
| Cystine | Cystine Impurity | | | | | | | | |
| | ≤2.0% | | | | | | | | |
| Total | Sum All | 0.5 | 0.6 | 0.8 | 0.8 | 0.8 | 0.8 | 1.0 | 1.0 |
| Impurity | Reported | | | | | | | | |
| | ≥0.05% | | | | | | | | |

TABLE 14
Inverted 10-mL L-cysteine hydrochloride monohydrate
at 30° C. ± 2° C. 65% RH ± 5%

| | | Result (%) | | | | |
|---|---|---|---|---|---|---|
| | Acceptance | months | | | | |
| Test | Criteria | 0 | 3 | 6 | 9 | 12 |
| Assay | 85% to | 109.4 | 110.9 | 110.2 | 110.7 | 108.6 |
| | 115% | | | | | |
| | label claim | | | | | |
| Individual | Known: | 0.45 | 0.57 | 0.72 | 0.60 | 0.72 |
| Impurity: | ≥0.05% | | | | | |
| Cystine | Report | | | | | |
| | Results | | | | | |
| | Cystine | | | | | |
| | Impurity | | | | | |
| | ≤2.0% | | | | | |
| Total | Sum All | 0.5 | 0.7 | 0.9 | 0.7 | 0.9 |
| Impurity | Reported | | | | | |
| | ≥0.05% | | | | | |

TABLE 15
Inverted 10-mL L-cysteine hydrochloride monohydrate
at 40° C. ± 2° C. 75% RH ± 5%

| | | Result (%) | | | | |
|---|---|---|---|---|---|---|
| | Acceptance | months | | | | |
| Test | Criteria | 0 | 1 | 2 | 3 | 6 |
| Assay | 85% to | 109.4 | 108.6 | 108.0 | 110.4 | 110.9 |
| | 115% | | | | | |
| | label claim | | | | | |
| Individual | Known: | 0.45 | 0.54 | 0.53 | 0.54 | 0.69 |
| Impurity: | ≥0.05% | | | | | |
| Cystine | Report | | | | | |
| | Results | | | | | |
| | Cystine | | | | | |
| | Impurity | | | | | |
| | ≤2.0% | | | | | |
| Total | Sum All | 0.5 | 0.7 | 0.7 | 0.8 | 0.9 |
| Impurity | Reported | | | | | |
| | ≥0.05% | | | | | |

TABLE 16
Upright 50-mL L-cysteine hydrochloride monohydrate at 25° C. ± 2° C. 60% RH ± 5%

| | | Result (%) | | | | | | | |
|---|---|---|---|---|---|---|---|---|---|
| | Acceptance | months | | | | | | | |
| Test | Criteria | 0 | 3 | 6 | 9 | 12 | 18 | 24 | 36 |
| Assay | 85% to 115% | 109.6 | 109.0 | 109.9 | 109.5 | 108.7 | 108.1 | 108.3 | 107.8 |
| | label claim | | | | | | | | |
| Individual | Known: ≥0.05% | 0.83 | 1.01 | 0.95 | 1.00 | 0.89 | 0.86 | 0.95 | 0.80 |
| Impurity: | Report Results | | | | | | | | |
| Cystine | Cystine Impurity | | | | | | | | |
| | ≤2.0% | | | | | | | | |
| Total | Sum All | 0.9 | 1.1 | 1.0 | 1.1 | 1.0 | 1.0 | 1.1 | 1.0 |
| Impurity | Reported | | | | | | | | |
| | ≥0.05% | | | | | | | | |

TABLE 17
Inverted 50-mL L-cysteine hydrochloride monohydrate at 25° C. ± 2° C. 60% RH ± 5%

| | | Result (%) | | | | | | | |
|---|---|---|---|---|---|---|---|---|---|
| | Acceptance | months | | | | | | | |
| Test | Criteria | 0 | 3 | 6 | 9 | 12 | 18 | 24 | 36 |
| Assay | 85% to 115% | 109.6 | 109.7 | 109.0 | 108.3 | 108.7 | 109.0 | 108.0 | 108.6 |
| | label claim | | | | | | | | |
| Individual | Known: ≥0.05% | 0.83 | 0.92 | 0.93 | 1.00 | 1.07 | 0.99 | 0.86 | 1.08 |
| Impurity: | Report Results | | | | | | | | |
| Cystine | Cystine Impurity | | | | | | | | |
| | ≤2.0% | | | | | | | | |
| Total | Sum All | 0.9 | 1.0 | 1.0 | 1.1 | 1.2 | 1.1 | 1.0 | 1.2 |
| Impurity | Reported | | | | | | | | |
| | ≥0.05% | | | | | | | | |

TABLE 18
Inverted 50-mL L-cysteine hydrochloride monohydrate
at 30° C. ± 2° C. 65% RH ± 5%

| | | Result (%) | | | | |
|---|---|---|---|---|---|---|
| | Acceptance | months | | | | |
| Test | Criteria | 0 | 3 | 6 | 9 | 12 |
| Assay | 85% to | 109.6 | 106.9 | 109.3 | 108.0 | 108.8 |
| | 115% | | | | | |
| | label claim | | | | | |
| Individual | Known: | 0.83 | 0.83 | 1.00 | 0.91 | 1.02 |
| Impurity: | ≥0.05% | | | | | |
| Cystine | Report | | | | | |
| | Results | | | | | |
| | Cystine | | | | | |
| | Impurity | | | | | |
| | ≤2.0% | | | | | |
| Total | Sum All | 0.9 | 1.0 | 1.1 | 1.0 | 1.2 |
| Impurity | Reported | | | | | |
| | ≥0.05% | | | | | |

TABLE 19
Inverted 50-mL L-cysteine hydrochloride monohydrate
at 40° C. ± 2° C. 75% RH ± 5%

| | | Result (%) | | | | |
|---|---|---|---|---|---|---|
| | Acceptance | months | | | | |
| Test | Criteria | 0 | 1 | 2 | 3 | 6 |
| Assay | 85% to | 109.6 | 108.5 | 107.4 | 107.9 | 108.4 |
| | 115% | | | | | |
| | label claim | | | | | |
| Individual | Known: | 0.83 | 1.02 | 0.89 | 0.97 | 1.18 |
| Impurity: | ≥0.05% | | | | | |
| Cystine | Report | | | | | |
| | Results | | | | | |
| | Cystine | | | | | |
| | Impurity | | | | | |
| | ≤2.0% | | | | | |
| Total | Sum All | 0.9 | 1.2 | 1.1 | 1.2 | 1.4 |
| Impurity | Reported | | | | | |
| | ≥0.05% | | | | | |

TABLE 20
Upright 50-mL L-cysteine hydrochloride monohydrate at 25° C. ± 2° C. 60% RH ± 5%

| | | Result (%) | | | | | | | |
|---|---|---|---|---|---|---|---|---|---|
| | Acceptance | months | | | | | | | |
| Test | Criteria | 0 | 3 | 6 | 9 | 12 | 18 | 24 | 36 |
| Assay | 85% to 115% | 107.9 | 108.7 | 109.5 | 107.6 | 107.8 | 108.0 | 107.5 | 108.1 |
| | label claim | | | | | | | | |
| Individual | Known: ≥0.05% | 0.87 | 0.82 | 0.80 | 0.99 | 1.03 | 0.93 | 0.89 | 0.83 |
| Impurity: | Report Results | | | | | | | | |
| Cystine | Cystine Impurity | | | | | | | | |
| | ≤2.0% | | | | | | | | |
| Total | Sum All | 0.9 | 0.9 | 0.9 | 1.1 | 1.1 | 1.1 | 1.0 | 1.0 |
| Impurity | Reported | | | | | | | | |
| | ≥0.05% | | | | | | | | |

TABLE 21
Inverted 50-mL L-cysteine hydrochloride monohydrate at 25° C. ± 2° C. 60% RH ± 5%

| | | Result (%) | | | | | | | |
|---|---|---|---|---|---|---|---|---|---|
| | Acceptance | months | | | | | | | |
| Test | Criteria | 0 | 3 | 6 | 9 | 12 | 18 | 24 | 36 |
| Assay | 85% to 115% | 107.9 | 106.9 | 110.6 | 108.6 | 108.1 | 108.6 | 107.6 | 106.4 |
| | label claim | | | | | | | | |
| Individual | Known: ≥0.05% | 0.87 | 0.82 | 0.99 | 0.99 | 1.10 | 0.95 | 0.94 | 1.1 |
| Impurity: | Report Results | | | | | | | | |
| Cystine | Cystine Impurity | | | | | | | | |
| | ≤2.0% | | | | | | | | |
| Total | Sum All | 0.9 | 0.9 | 1.1 | 1.1 | 1.2 | 1.1 | 1.1 | 1.3 |
| Impurity | Reported | | | | | | | | |
| | ≥0.05% | | | | | | | | |

TABLE 22
Inverted 50-mL L-cysteine hydrochloride monohydrate
at 30° C. ± 2° C. 65% RH ± 5%

| | | Result (%) | | | | |
|---|---|---|---|---|---|---|
| | Acceptance | months | | | | |
| Test | Criteria | 0 | 3 | 6 | 9 | 12 |
| Assay | 85% to 115% | 107.9 | 108.6 | 109.6 | 108.3 | 108.1 |
| | label claim | | | | | |
| Individual | Known: ≥0.05% | 0.87 | 0.96% | 0.98% | 0.99% | 1.11% |
| Impurity: | Report Results | | | | | |
| Cystine | Cystine Impurity | | | | | |
| | ≤2.0% | | | | | |
| Total | Sum All | 0.9 | 1.1 | 1.1 | 1.1 | 1.2 |
| Impurity | Reported | | | | | |
| | ≥0.05% | | | | | |

TABLE 23
Inverted 50-mL L-cysteine hydrochloride monohydrate
at 40° C. ± 2° C. 75% RH ± 5%

| | | Result (%) | | | | |
|---|---|---|---|---|---|---|
| | Acceptance | months | | | | |
| Test | Criteria | 0 | 1 | 2 | 3 | 6 |
| Assay | 85% to | 107.9 | 107.5 | 106.6 | 109.0 | 109.5 |
| | 115% | | | | | |
| | label claim | | | | | |
| Individual | Known: | 0.87 | 0.94 | 0.94 | 1.10 | 1.07 |
| Impurity: | ≥0.05% | | | | | |
| Cystine | Report | | | | | |
| | Results | | | | | |
| | Cystine | | | | | |
| | Impurity | | | | | |
| | ≤2.0% | | | | | |
| Total | Sum All | 0.9 | 1.1 | 1.1 | 1.3 | 1.3 |
| Impurity | Reported | | | | | |
| | ≥0.05% | | | | | |

TABLE 24
Upright 50-mL L-cysteine hydrochloride monohydrate at 25° C. ± 2° C. 60% RH ± 5%

| | | Result (%) | | | | | | | |
|---|---|---|---|---|---|---|---|---|---|
| | Acceptance | months | | | | | | | |
| Test | Criteria | 0 | 3 | 6 | 9 | 12 | 18 | 24 | 36 |
| Assay | 85% to 115% | 107.5 | 109.2 | 110.4 | 108.9 | 107.6 | 107.9 | 107.4 | 108.0 |
| | label claim | | | | | | | | |
| Individual | Known: ≥0.05% | 1.00 | 0.92 | 1.01 | 0.92 | 1.01 | 0.93 | 0.92 | 0.83 |
| Impurity: | Report Results | | | | | | | | |
| Cystine | Cystine Impurity | | | | | | | | |
| | ≤2.0% | | | | | | | | |
| Total | Sum All | 1.1 | 1.0 | 1.1 | 1.0 | 1.1 | 1.1 | 1.1 | 1.0 |
| Impurity | Reported | | | | | | | | |
| | ≥0.05% | | | | | | | | |

TABLE 25
Inverted 50-mL L-cysteine hydrochloride monohydrate at 25° C. ± 2° C. 60% RH ± 5%

| | | Result (%) | | | | | | | |
|---|---|---|---|---|---|---|---|---|---|
| | Acceptance | months | | | | | | | |
| Test | Criteria | 0 | 3 | 6 | 9 | 12 | 18 | 24 | 36 |
| Assay | 85% to 115% | 107.5 | 108.6 | 109.7 | 107.9 | 107.5 | 108.6 | 107.5 | 107.9 |
| | label claim | | | | | | | | |
| Individual | Known: ≥0.05% | 1.00 | 0.96 | 1.00 | 1.02 | 1.07 | 0.94 | 0.92 | 0.96 |
| Impurity | Report Results | | | | | | | | |
| Cystine | Cystine Impurity | | | | | | | | |
| | ≤2.0% | | | | | | | | |
| Total | Sum All | 1.1 | 1.1 | 1.1 | 1.1 | 1.2 | 1.1 | 1.1 | 1.2 |
| Impurity | Reported | | | | | | | | |
| | ≥0.05% | | | | | | | | |

TABLE 26
Inverted 50-mL L-cysteine hydrochloride monohydrate
at 30° C. ± 2° C. 65% RH ± 5%

| | | Result (%) | | | | |
|---|---|---|---|---|---|---|
| | Acceptance | months | | | | |
| Test | Criteria | 0 | 3 | 6 | 9 | 12 |
| Assay | 85% to | 107.5 | 109.1 | 111.0 | 108.2 | 108.8 |
| | 115% | | | | | |
| | label claim | | | | | |
| Individual | Known: | 1.00 | 0.97 | 0.97 | 1.06 | 0.93 |
| Impurity: | ≥0.05% | | | | | |
| Cystine | Report | | | | | |
| | Results | | | | | |
| | Cystine | | | | | |
| | Impurity | | | | | |
| | ≤2.0% | | | | | |
| Total | Sum All | 1.1 | 1.1 | 1.1 | 1.2 | 1.0 |
| Impurity | Reported | | | | | |
| | ≥0.05% | | | | | |

TABLE 27
Inverted 50-mL L-cysteine hydrochloride monohydrate
at 40° C. ± 2° C. 75% RH ± 5%

| | | Result (%) | | | | |
|---|---|---|---|---|---|---|
| | Acceptance | months | | | | |
| Test | Criteria | 0 | 1 | 2 | 3 | 6 |
| Assay | 85% to | 107.5 | 107.2 | 106.7 | 108.6 | 108.9 |
| | 115% | | | | | |
| | label claim | | | | | |
| Individual | Known: | 1.00 | 0.88 | 0.96 | 1.03 | 1.13 |
| Impurity: | ≥0.05% | | | | | |
| Cystine | Report | | | | | |
| | Results | | | | | |
| | Cystine | | | | | |
| | Impurity | | | | | |
| | ≤2.0% | | | | | |
| Total | Sum All | 1.1 | 1.0 | 1.1 | 1.2 | 1.3 |
| Impurity | Reported | | | | | |
| | ≥0.05% | | | | | |

### Results

As shown in Tables 6-27, the 10-mL and 50-mL drug product containing 50 mg/mL L-cysteine hydrochloride monohydrate and WFI was stable at all storage condition and durations. The Cystine Impurity levels were also stable across all the stability time points. Thus, drug product produced by the process of the present invention is free of the sterilization issues that accompany heat sensitive active pharmaceutical ingredients.

### Example 4—Effect of Autoclaving in the Presence of Oxygen on Stability of L-Cysteine Hydrochloride Monohydrate Produced by a Process of the Invention

### Purpose of the Experiment

The purpose of this experiment was to determine whether the L-cysteine hydrochloride monochloride produced by a process of the invention is amenable to terminal sterilization.

### Methods

When aqueous L-Cysteine HCl solution is exposed to oxygen, it may oxidize readily to give cystine at neutral or basic pH. Stock solutions of cysteine are relatively stable at acidic pH, especially in degassed solutions. Cysteine is more stable in acidic solutions.

In this study, L-Cysteine HCl injection drug product prepared by a process of Example 1 of the invention, was exposed to air for 10 minutes and then was autoclaved for 8 minutes at 121° C. L-Cysteine HCl assay and Cystine Impurity Assay were measured and reported. The results are summarized in Table 28.

### Results

TABLE 28
L-Cysteine HCl injection drug product vs L-Cysteine HCl injection drug product
exposed to air for 10 minutes

| | | Result (%) | | | | | | | |
|---|---|---|---|---|---|---|---|---|---|
| | | | | | | L-Cysteine HCl injection drug | | | |
| | | L-Cysteine HCl injection drug | | | | product exposed to air for 10 | | | |
| | | product (“as is” product) | | | | minutes | | | |
| | | Arm 1 | | Arm 1 | | Arm 2 | | Arm 2 | |
| | | Unsterilized | | Sterilized | | Unsterilized | | Sterilized | |
| | Acceptance | Group | Group | Group | Group | Group | Group | Group | Group |
| Test | Criteria | A-1 | A-2 | B-1 | B-2 | A-1 | A-2 | B-1 | B-2 |
| Assay | 85% to | 99.6 | 99.9 | 100.1 | 100.1 | 99.1 | 100.4 | 99.0 | 99.0 |
| | 115% label | | | | | | | | |
| | claim | | | | | | | | |
| Impurities | Cystine: ≤2.0% | 0.28 | 0.41 | 0.26 | 0.30 | 0.64 | 0.38 | 0.79 | 0.89 |

| | | | | | |
|---|---|---|---|---|---|
| Assay | 85% to | 99.75% | 100.1% | 99.75% | 99.0% |
| (average) | 115% label | | | | |
| | claim | | | | |
| Impurity | Cystine: ≤2.0% | 0.35% | 0.28% | 0.51% | 0.84% |
| (average) | | | | | |

For the “as is” product, sterilizing the product did not change the cystine impurity levels. Further, when the L-cysteine HCl Injection product was exposed to air for 10 minutes, the cystine impurity level went up from 0.35% to 0.51%.

When the “as is” product was exposed to air for 10 minutes and then steam sterilized, the cystine impurity level went up from 0.35% to 0.84% (2.4 times).

The results of this experiment suggest that exposing the L-Cysteine HCl injection drug product prepared by processes of the invention to oxygen, and then sterilizing the drug product, leads to degradation of L-Cysteine HCl drug, as indicated by elevated levels of cystine impurity (Oxidative degradation product of L-Cysteine HCl).

The experiment demonstrates that the L-Cysteine HCl Injection Drug Product was amenable to terminal sterilization due to maintenance of very low levels of oxygen in the liquid drug product and head space.

What is claimed is:

1. A stable liquid formulation comprising L-cysteine hydrochloride, wherein the formulation is amenable to terminal sterilization by heat, wherein the formulation contains no more than 10 parts per million (ppm) of dissolved oxygen.

2. A stable sterile liquid formulation comprising L-cysteine hydrochloride, wherein the formulation was terminally sterilized by heat.

3. The formulation of claim 2, wherein the formulation is substantially free of oxygen.

4. The formulation of claim 3, wherein the formulation contains no more than 10 parts per million (ppm) of dissolved oxygen.

5. The formulation of claim 1, wherein the formulation is in a form of a sterile non-pyrogenic parenteral solution.

6. The formulation of claim 1, further comprising one or more of pharmaceutically acceptable excipients.

7. The formulation of claim 6, wherein the excipient comprises one or more of hydrochloric acid, sodium hydroxide and water.

8. The formulation of claim 2, wherein the formulation when stored at a storage temperature of between 20° C. and 40° C. for three months contains less than about 2% of cystine impurities.

9. The formulation of claim 2, wherein the formulation when stored at a storage temperature of between 20° C. and 40° C. for six months contains less than about 2% of cystine impurities.

10. The formulation of claim 2, wherein the formulation when stored at a storage temperature of between 20° C. and 40° C. for nine months contains less than about 2% of cystine impurities.

11. The formulation of claim 2, wherein the formulation when stored at a storage temperature of between 20° C. and 40° C. for twelve months contains less than about 2% of cystine impurities.

12. The formulation of claim 8, wherein the formulation contains less than about 1% of cystine impurities.

13. The formulation of claim 9, wherein the formulation contains less than about 1% of cystine impurities.

14. The formulation of claim 10, wherein the formulation contains less than about 1% of cystine impurities.

15. The formulation of claim 11, wherein the formulation contains less than about 1% of cystine impurities.

16. The formulation of claim 8, wherein the storage temperature is between about 20° C. and about 25° C.

17. The formulation of claim 1, wherein the composition has a pH from about 1.0 to about 2.5.

18. A container comprising the formulation of claim 1, wherein the container is a single dose vial.

19. The container of claim 18 wherein the formulation is steam sterilized by heat.

20. A process of manufacturing a terminally steam sterilized pharmaceutical composition, wherein the composition comprises L-cysteine hydrochloride, the process comprising the following steps:
(i) adding a pharmaceutically acceptable vehicle to a batch container comprising:
(a) a top side comprising an opening; and
(b) a pressurizing means,
(ii) purging the batch container with nitrogen gas;
(iii) mixing the drug vehicle;
(iv) adding L-cysteine hydrochloride monohydrate to the pharmaceutically acceptable vehicle to create the pharmaceutical composition;
(v) mixing the pharmaceutical composition until the L-cysteine hydrochloride monohydrate is dissolved in the pharmaceutically acceptable vehicle;
(vi) sealing the opening of the top side of the batch container;
(vii) pressurizing the batch container with nitrogen gas;
(viii) optionally, sterilizing stoppers, preferably in sterilization bags, preferably at about 121° C. and preferably for 30 minutes;
(ix) optionally, sterilizing filling equipment, preferably at about 121° C., preferably for 30 minutes, and preferably wrapping filling equipment prior to sterilization;
(x) optionally, washing vials with water for injection and depyrogenating vials in a depyrogenation oven;
(xi) optionally, filling a product container with the pharmaceutical composition from the batch container using filling equipment;
(xii) optionally, stoppering the product container; and
(xiii) optionally, capping the product container.

21. A sterile liquid pharmaceutical composition prepared by a process of claim 20.
